(19) United States
(12) Patent Application Publication
(10) Pub. No.: US 2019/0117294 A1
(43) Pub. Date: Apr. 25, 2019

(54) GRASPING TREATMENT INSTRUMENT

(71) Applicant: Olympus Corporation, Tokyo (JP)

(72) Inventors: Akihito Kano, Tokyo (JP); Mizuki Komiya, Tokyo (JP); Ryuhei Shimada, Tokyo (JP)

(73) Assignee: Olympus Corporation, Tokyo (JP)

(21) Appl. No.: 16/195,381

(22) Filed: Nov. 19, 2018

Related U.S. Application Data
(63) Continuation of application No. PCT/JP2016/065485, filed on May 25, 2016.

(51) Int. Cl. A61B 18/14

(52) U.S. Cl. CPC A61B 18/1445; A61B 2017/2925

(57) ABSTRACT

A grasping treatment instrument can be opened and closed between a first grasping jaw and a second grasping jaw. The first grasping jaw includes, on its outer front surface, a grasping surface opposing the second grasping jaw, a back surface facing an opposite side to the grasping surface, and inclined surfaces arranged extending from a side of the back surface toward a side of the grasping surface as the inclined surfaces become away from a central position in a width direction, and are inclined relative to the back surface. In the first grasping jaw, a liquid is supplied from a liquid inlet portion onto the inclined surfaces by way of the back surface, and the liquid flows out from the inclined surfaces toward the side of the grasping surface.

### CROSS-REFERENCE TO RELATED APPLICATION

This application is a continuation application of PCT Application No. PCT/JP2016/065485 filed on May 25, 2016, which is hereby incorporated by reference in its entirety.

### TECHNICAL FIELD

The disclosed technology generally relates to a grasping treatment instrument, which grasps a treatment object between a pair of grasping jaws and treats the grasped treatment object.

### DESCRIPTION OF THE RELATED ART

US 2007/0049920 A1 discloses a surgical device, in other words, a grasping treatment instrument treating a treatment object such as biological tissue by grasping the treatment object between a pair of grasping jaws and causing a high frequency electric current to flow through the grasped treatment object. In the treatment with a high frequency electric current, a treatment object may be heated to high temperature. In this case, there is a possibility that the treatment object may stick to the grasping jaws or may undergo char formation.

There is a need for a grasping treatment instrument which ensures the supply performance of a liquid to a treatment object and its vicinity and effectively prevents sticking, char formation, or the like of the treatment object.

### BRIEF SUMMARY OF EMBODIMENTS

One aspect of the disclosed technology is directed to a grasping treatment instrument comprises a first grasping jaw having opposed respective proximal and distal end portions and an outer front surface. A second grasping jaw is configured to be engaged with first grasping jaw so as to relatively pivot with respect to one another. The first grasping jaw includes a grasping surface formed on the outer front surface and opposing the second grasping jaw. Aback surface is formed on the outer front surface and facing an opposite side to the grasping surface. A first recessed portion is formed in the back surface toward a side of the grasping surface and forming a first flow path on which a liquid flows from the proximal end portion toward the distal end portion of the first grasping jaw. A second recessed portion is formed in the outer front surface toward the side of the grasping surface extending in continuation with the first recessed portion and in a direction intersecting the first recessed portion and forming a second flow path on which the liquid flows toward the side of the grasping surface. A liquid port is formed through the proximal end portion of the first grasping jaw so that the liquid flows into the first recessed portion in the back surface.

Another aspect of the disclosed technology is directed to A grasping treatment instrument comprises a first grasping jaw having opposed respective proximal and distal end portions and an outer front surface. A second grasping jaw is configured to be engaged with first grasping jaw so as to relatively pivot with respect to one another. The first grasping jaw includes a grasping surface formed on the outer front surface and opposing the second grasping jaw. Aback surface is formed on the outer front surface and facing an opposite side to the grasping surface. A first flow path is formed on the back surface to direct liquid to flow from the proximal end portion toward the distal end portion. A second flow path is formed on the outer front surface and having inclined surfaces that are formed extending from a side of the back surface toward a side of the grasping surface as the inclined surfaces become away from a central position in a width direction of the first grasping jaw and are inclined relative to the back surface. An adjacent surface is formed adjacent to the first flow path and the second flow path. A liquid port is formed through the proximal end portion of the first grasping jaw so that the liquid flow to the first flow path in the back surface. The first flow path has a surface having higher hydrophilicity than the adjacent surface.

A further aspect of the disclosed technology is directed to a method of using grasping treatment instrument for treating liver parenchyma or a liver blood vessel. The method comprises the steps of grasping the liver parenchyma or the liver blood vessel by respective first and second grasping jaws simultaneously in an abdominal cavity. Next, applying a high frequency electric current to flow across an electrode, which is disposed in the first grasping jaw, and the second grasping jaw to apply the high frequency electric current to the liver parenchyma or the liver blood vessel. Next, supplying a liquid to a back surface, which is formed in an outer front surface of the first grasping jaw that faces an opposite side to the electrode, before or concurrently with applying the high frequency electric current. Finally, directing the liquid to flow by way of a first recessed portion and a second recessed portion that is disposed in continuation with the first recessed portion and extending in a direction intersecting the first recessed portion, and to flow out toward a side of the electrode.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a schematic view illustrating a treatment system according to a first embodiment.

FIG. 2 is a cross-sectional view schematically depicting a first grasping jaw in the first embodiment in a cross-section substantially perpendicular to a width direction.

FIG. 3 is a cross-sectional view taken along a line of FIG. 2.

FIG. 4 is a cross-sectional view taken along a line IV-IV of FIG. 2.

FIG. 5 is a perspective view schematically depicting a configuration of a distal end portion of a support member in the first grasping jaw in the first embodiment.

FIG. 6 is a schematic view illustrating wetting of a bottom surface of a first recessed portion or a bottom surface of a second recessed portion in a first grasping jaw in a first modification.

FIG. 7 is a schematic view illustrating a configuration of a bottom surface of a first recessed portion or a bottom surface of a second recessed portion in a first grasping jaw in a second modification.

FIG. 8 is a schematic view illustrating wetting of an adjacent surface in a first grasping jaw in a third modification.

FIG. 9 is a perspective view schematically depicting a configuration of a distal end portion of a support member in a first grasping jaw in a fourth modification.

FIG. 10 is a perspective view schematically depicting a configuration of a distal end portion of a support member in a first grasping jaw in a fifth modification.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

### First embodiment

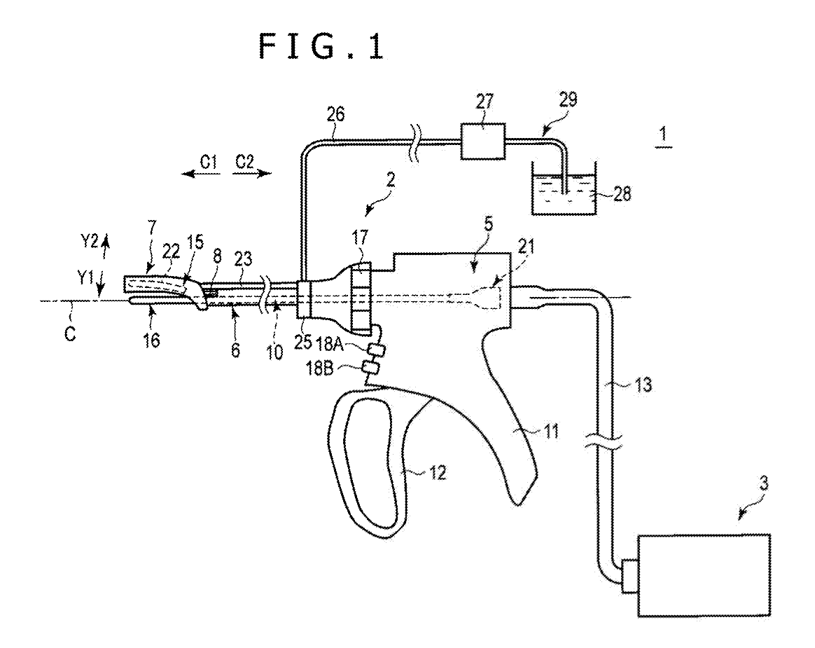

Referring to FIGS. 1 through 5, a description will be made about a first embodiment of the present disclosure.

FIG. 1 is a view illustrating a treatment system 1 according to this embodiment. As illustrated in FIG. 1, the treatment system 1 includes a grasping treatment instrument 2 and an energy control system 3. The grasping treatment instrument 2 has a longitudinal axis C. Here, a side in a direction along the longitudinal axis C is assumed to be a distal end side or the side indicated by an arrow Cl, while a side opposite to the distal end side is assumed to be a proximal end side or the side indicated by an arrow C2.

The grasping treatment instrument 2 also includes a hand-holdable housing 5, a shaft or sheath 6 connected to a distal end side of the housing 5, and an end effector 7 disposed on a distal end portion of the shaft 6. The shaft 6 is disposed centering substantially around the longitudinal axis C and extending along the longitudinal axis C. On the housing 5, a grip or a fixed handle 11 is arranged, and a handle or a movable handle 12 is pivotally attached. The handle 12 is pivoted relative to the housing 5, whereby the handle 12 is opened or closed with respect to the grip 11.

The end effector 7 includes a first grasping jaw 15 and a second grasping jaw 16. The first grasping jaw 15 is pivotally attached to the distal end portion of the shaft 6. Inside the shaft 6, a movable member 8 is disposed extending along the longitudinal axis C. Inside the housing 5, the movable member 8 is connected to the handle 12. Also, the movable member 8 is connected at a distal end portion thereof to the first grasping jaw 15. By opening or closing the handle 12 with respect to the grip 11, the movable member 8 is driven so that the movable member 8 is allowed to move relative to the housing 5 and the shaft 6 along the longitudinal axis C. As a consequence, the first grasping jaw 15 pivots about the position of its attachment to the shaft 6, so that the first grasping jaw 15 is opened or is closed with respect to the second grasping jaw 16. Accordingly, the paired grasping jaws 15 and 16 open or close. By closing the paired grasping jaws 15 and 16, a treatment object such as biological tissue can be grasped between the grasping jaws 15 and 16. The open/close direction of the first grasping jaw 15, in other words, the direction indicated by an arrow Y1 and an arrow Y2 intersects the longitudinal axis C, in other words, is substantially perpendicular to the longitudinal axis C.

The second grasping jaw 16 may be integral with the shaft 6 or may be fixed to the shaft 16, or may be pivotally attached to the shaft 6. In a case in which the second grasping jaw 16 is pivotally attached to the shaft 6, the movable member 8 is moved along the longitudinal axis C, whereby the second grasping jaw 16 in addition to the first grasping jaw 15 is also pivoted relative to the shaft 6 and the grasping jaws 15 and 16 hence open or close. Also, in certain examples, a rod member which is, for example, designated at numeral reference 10 may be disposed, specifically inserted through the shaft 6, and the second grasping jaw 16 may be formed by a portion of the rod member 10, the portion protruding from the shaft 6 toward the distal end side. Moreover, in this embodiment, a turnable knob 17 is attached to the housing 5. By turning the turnable knob 17 relative to the housing 5, the shaft 6, the end effector 7, and the movable member 8 are turned together with the turnable knob 17 about the longitudinal axis C or a central axis of the shaft 6.

A cable 13 is connected at an end thereof to the housing 5. The cable 13 is connected at an opposite end thereof to the energy control system 3. To the housing 5, control buttons 18A and 18B are also attached as energy control input portions. By independently pressing the control buttons 18A and 18B, control is input to the energy control system 3 in order to output electric energy from the energy control system 3 to the grasping treatment instrument 2. In place of or in addition to the control buttons 18A and 18B, a footswitch or the like, which is discrete from the grasping treatment instrument 2, may be included as an energy control input portion.

The energy control system 3 includes a power source such as a battery or a power outlet, a conversion circuit, a control unit such as a processor or an integrated circuit, and a storage medium. The conversion circuit converts electric power from the power source to electric energy to be supplied to the grasping treatment instrument. The control unit includes a central processing unit (CPU), an application specific integrated circuit (ASIC), or the like. Based on a control input through the control button 18A or the energy control input portion, the energy control system 3 outputs high frequency electric energy as electric energy. The high frequency electric energy output from the energy control system 3 is supplied to the first grasping jaw 15 and the second grasping jaw 16.

In certain examples, the second grasping jaw 16 is formed by the portion of the rod member 10 described hereinbefore, the portion protruding from the shaft 6, and inside the housing 5, an ultrasonic transducer 21 is connected to a proximal end side of the rod member 10. When a control input is performed through the control button 18B, high frequency electric energy is supplied to the grasping jaws 15 and 16 from the energy control system 3, and at the same time, electric energy different from the high frequency electric energy supplied to the grasping jaws 15 and 16, for example, AC power of a predetermined frequency is supplied from the energy control system 3 to the ultrasonic transducer 21. As a consequence, ultrasonic vibrations occur at the ultrasonic transducer 21. The ultrasonic vibrations occurred at the ultrasonic transducer 21 are transmitted to the second grasping jaw 16 by way of the rod member 10. Consequently, the rod member 10 including the second grasping jaw 16 is caused to resonate or vibrate, whereby ultrasonic vibrations are applied as treatment energy to the treatment object grasped between the grasping jaws 15 and 16.

In some different examples, a heating element 22 may be disposed in at least one of the grasping jaws 15 and 16, for example, the first grasping jaw 15. When a control input is performed through the control button 18B, high frequency electric energy is supplied to the grasping jaws 15 and 16 from the energy control system 3, and at the same time, electric energy different from the high frequency electric energy supplied to the grasping jaws 15 and 16, for example, DC electric power or AC electric power is supplied from the energy control system 3 to the heating element 22. As a consequence, heat is generated at the heating element 22, and the generated heat is applied as treatment energy to the grasped treatment object.

On an outer peripheral surface of the shaft 6, a feed tube 23 is disposed extending along the longitudinal axis C. The feed tube 23 is connected at an end thereof, specifically a distal end thereof to the first grasping jaw 15. On a distal end side of the turnable knob 17, a coupler member 25 is fixed. The feed tube 23 is connected at an opposite end thereof, specifically a proximal end thereof to the fluid coupler 25. The feed tube 23 and coupler member 25 are turnable together with the turnable knob 17 and the shaft 6 relative to the housing 5 about the longitudinal axis C.

An external tube 26 is connected at an end thereof to the coupler member 25. In the coupler member 25, the feed tube 23 and the external tube 26 internally communicate to each other. The external tube 26 is connected at an opposite end thereof to a liquid source 29 that includes a feed pump 27 and a liquid tank 28. When the feed pump 27 is driven, a liquid such as physiological saline stored in the liquid tank 28 is supplied through the inside of the external tube 26. Along a feed route inside the feed tube 23, the liquid is then supplied or fed from the proximal end side to the distal end side. In certain examples, the feed tube 23 may be disposed extending along the longitudinal axis C inside the shaft 6, and the liquid may be supplied from the proximal end side toward the distal end side through the feed route inside the feed tube 23. In some different examples, a multi-lumen tube, not illustrated, may be disposed extending along the longitudinal axis C. In such a case, the shaft 6 is inserted through one of lumens in the multi-lumen tube, and another lumen is used as a feed route supplying the liquid from the proximal end side to the distal end side.

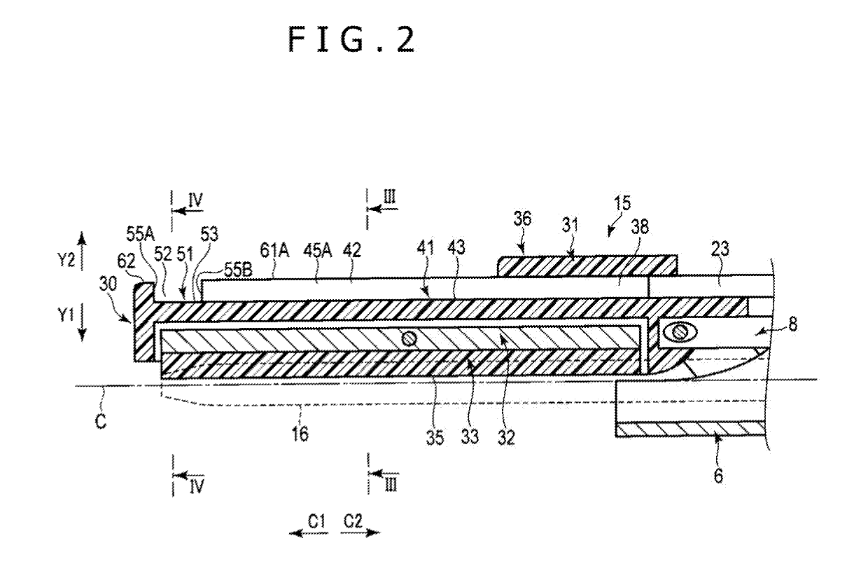

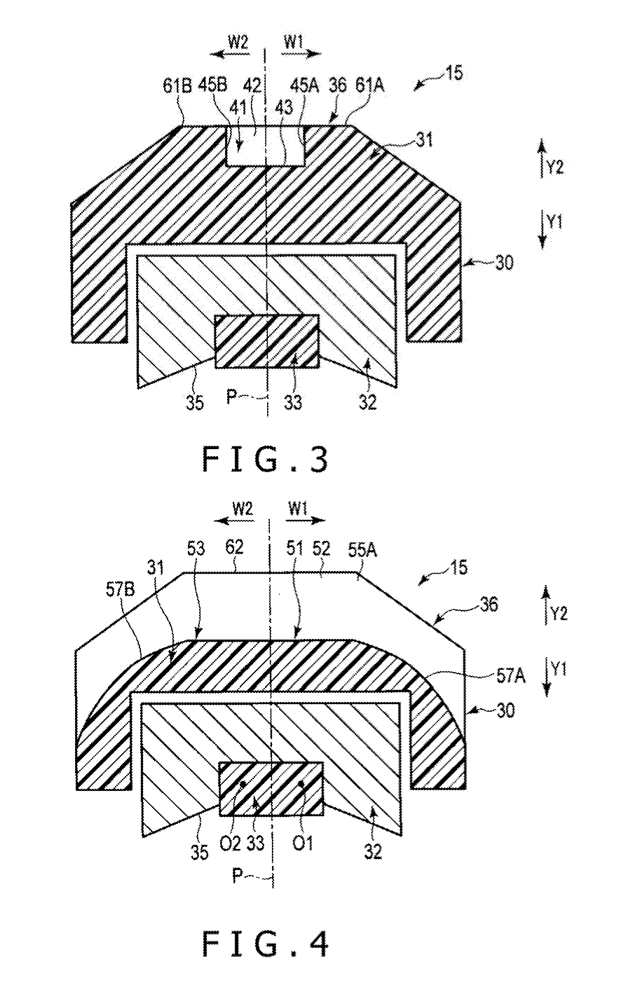

FIGS. 2 to 4 depict the configuration of the first grasping jaw 15. Here, a direction, which intersects at substantially right angles the longitudinal axis C, in other words, the direction of extending disposition of the first grasping jaw 15 and also intersects, in other words, is substantially perpendicular to the open/close direction of the first grasping jaw 15, in other words, directions indicated by the arrow Y1 and the arrow Y2, is assumed to be a width direction of the first grasping jaw 15 or the end effector 7, that is, a direction indicated by an arrow W1 and an arrow W2. FIG. 2 depicts the first grasping jaw 15 in a cross-section substantially perpendicular to the width direction. FIG. 3 depicts a cross-section taken along a line of FIG. 2, and FIG. 4 depicts a cross-section taken along a line IV-IV of FIG. 2. Therefore, FIGS. 3 and 4 depict the first grasping jaw 15 in cross-section substantially perpendicular to the direction along the longitudinal direction C, and the cross-section of FIG. 4 is taken at a position closer to the distal end side than the cross-section of FIG. 3.

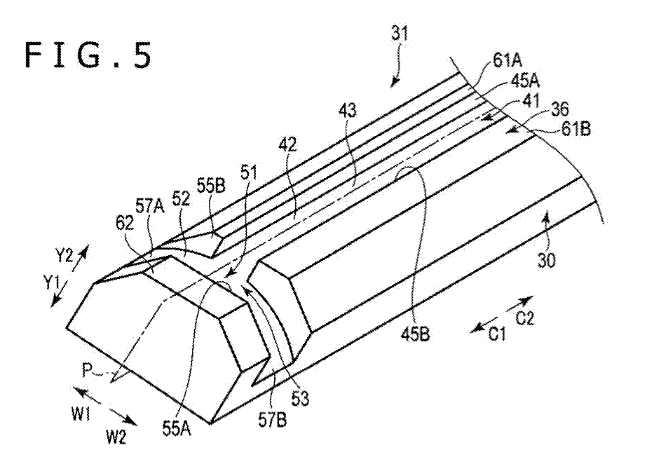

As depicted in FIGS. 2 to 4, the first grasping jaw 15 has a proximal end and a distal end, and is disposed extending from the proximal end to the distal end along the longitudinal axis C. The first grasping jaw 15 includes a support member 31, an electrode 32, and a pad member 33. The support member 31, the electrode 32, and the pad member 33 are disposed extending over from a proximal end portion to a distal end portion of the first grasping jaw 15. The electrode 32 is formed from an electrically conductive material, while the pad member 33 is formed from an electrically insulating material. Preferably, the support member 31 may have coating applied to a surface thereof with an electrically insulating material. The electrode 32 and the pad member 33 may be pivotally secured on the support member 31 like a so-called seesaw jaw or wiper jaw, or may be fixedly secured on the support member 31. FIG. 5 depicts a configuration of a distal end portion of the support member 31.

As depicted in FIGS. 2 through 5, the first grasping jaw 15 has an outer front surface 30 exposed to an outside. In this embodiment, by the electrode 32 and the pad member 33, a grasping surface 35 is formed on the outer front surface 30 of the first grasping jaw 15 such that the grasping surface 35 faces a side toward which the first grasping jaw 15 is closed, in other words, the side indicated by the arrow Yl, and by the support member 31, a back surface 36 is formed on the outer front surface 30 of the first grasping jaw 15 such that the back surface 36 faces a side toward which the first grasping jaw 15 is opened, in other words, the side indicated by the arrow Y2. The grasping surface 35 opposes the second grasping jaw 16, so that the treatment object grasped between the grasping jaws 15 and 16 comes in contact with the grasping surface 35. On the other hand, the back surface 36 faces an opposite side to the grasping surface 35.

The support member 31 is pivotally attached to the shaft 6, and is connected to a distal end portion of the movable member 8 via a connecting pin 37. Therefore, the position of attachment of the support member 31 to the shaft 6 serves as a fulcrum for pivotal motion of the support member 31, in other words, the first grasping jaw 15, and the position of connection of the support member 31 to the movable member 8 serves as a point of effort where drive force acts to cause pivoting of the support member 31. A port 38, or a liquid port 38, is formed in the support member 31 of the first grasping jaw 15. In the port 38, the feed tube 23 is connected at an end thereof, specifically the distal end thereof to the support member 31 from the proximal end side, so that the port 38 communicates with the feed route inside the feed tube 23. In the examples in which the feed route is formed by one of the lumens in the multi-lumen tube, the port 38 communicates to the lumen of the multi-lumen tube, which is used as the feed route.

When high frequency electric energy is supplied to the grasping jaws 15 and 16 from the energy control system 3, different potentials occur at the electrode 32 in the first grasping jaw 15 and at the second grasping jaw 16, respectively. Therefore, a high frequency electric current flows through the treatment object between the electrode 32 and the second grasping jaw 16 when the high frequency electric energy is supplied to the grasping jaws 15 and 16 with the grasped treatment target being in contact with the electrode 32 and the second grasping jaw 16. As a consequence, the high frequency electric current is applied as treatment energy to the treatment target grasped between the grasping jaws 15 and 16.

A first recessed portion 41 or a first recessed surface is arranged in the back surface 36 of the first grasping jaw 15. The first recessed portion 41 or the first recessed surface is recessed toward the side of the grasping surface 35, in other words, the side toward which the first grasping jaw 15 is closed. More specifically, in the back surface 36 of the first grasping jaw 15, a liquid guide groove or a flow path is formed to allow the liquid to flow from the proximal end side toward the distal end side. The first recessed portion 41 is disposed extending from the proximal end portion to the distal end portion of the first grasping jaw 15 in the direction of extending disposition of the first grasping jaw 15. Here, a central position P or a central surface in the width direction of the first grasping jaw 15 will be specified (see FIGS. 3 to 5). In this embodiment, the first recessed portion 41 is located, over substantially the entire length thereof from its proximal end to its distal end, at the central position P in the width direction of the first grasping jaw 15 and in a vicinity of the central position P. As the first recessed portion 41 is disposed in the back surface 36, the first recessed portion 41 is exposed to the outside of the first grasping jaw 15. By the first recessed portion 41, a first recessed space 42 is formed opening toward the side toward which the first grasping jaw 15 is opened, in other words, the side indicated by the arrow Y2. The first recessed portion 41 has a bottom surface or a first bottom surface 43, and side surfaces or first side surfaces 45A and 45B. FIG. 3 depicts a cross-section of the first grasping jaw 15 taken along a plane that intersects the first recessed portion 41 and is substantially perpendicular to the direction of extending disposition of the first grasping jaw 15. Also, in certain examples, the first recessed portion 41 has a width, in other words, a dimension between the side surfaces 45A and 45B of 3 mm or smaller, and has a depth, in other words, a dimension from its opening to the bottom surface 43 of 1 mm or smaller.

The first recessed space 42 communicates with the port 38. The liquid, which has been supplied toward the distal end side along the feed route inside the feed tube 23, therefore flows from the port 38 into the first recessed space 42 in the back surface 36. Also, in the examples in which the feed route is formed by one of the lumens in the multi-lumen tube, the liquid which has been supplied through the lumen as the feed route in the multi-lumen tube flows into the first recessed space 42. Therefore, the port 38 is used as a liquid inlet portion that allows the liquid to flow onto the back surface 36 of the first grasping jaw 15 or into the first recessed space 42 in this embodiment. In the first recessed space 42, the flowed-in liquid flows from the proximal end side toward the distal end side. Here, the liquid flows along the bottom surface, specifically the first bottom surface 43 of the first recessed portion 41 formed in the back surface 36. Therefore, on the back surface 36 in this embodiment, a flow path surface on which the liquid flowed in from the port 38 flows from the proximal end side toward the distal end side is formed on the bottom surface 43 of the first recessed portion 41 or the first recessed surface. In certain examples, the liquid flows at a flow rate of 4 mL/min or lower on the bottom surface 43 or the flow path surface of the first recessed portion 41.

On the outer front surface 30 of the first grasping jaw 15, a second recessed portion 51 or a second recessed surface is disposed such that it is recessed toward the side of the grasping surface 35, in other words, the side toward which the first grasping jaw 15 is closed. The second recessed portion 51 is disposed on the distal end portion of the first grasping jaw 15, and the first recessed portion 41 continues at a distal end thereof to the second recessed portion 51. Therefore, the second recessed portion 51 is located on the distal end side relative to the first recessed portion 41, in other words, the bottom surface 43 that is used as a flow path surface. In this embodiment, the second recessed portion 51 is disposed extending from the central position P in the width direction of the first grasping jaw 15 toward opposite sides in the width direction of the first grasping jaw 15, in other words, towards the side indicated by the arrow W1 and the side indicated by the arrow W2. In other words, the second recessed portion 51 is disposed extending from the central position P or the central surface toward outer sides in the width direction of the first grasping jaw 15. As the second recessed portion 51 is disposed in the outer front surface 30, the second recessed portion 51 is exposed to the outside of the first grasping jaw 15. By the second recessed portion 51, a second recessed space 52 is formed opening toward the side toward which the first grasping jaw 15 is opened, in other words, the side indicated by the arrow Y2. The first recessed space 42 communicates at a distal end thereof to the second recessed space 52.

The second recessed portion 51 has a bottom surface 53 or a second bottom surface and side surfaces 55A and 55B or second side surfaces. The side surface 55A or the side surface on the distal end side forms a distal end of the second recessed portion 51, and faces the proximal end side. On the other hand, the side surface 55B or the side surface on the proximal end side forms a proximal end of the second recessed portion 51, and faces the distal end side. The side surface 55A or a wall surface is arranged continuously extending in the width direction of the first grasping jaw 15. On the other hand, the side surface 55B becomes discontinuous in the width direction of the first grasping jaw 15 at a communication part between the first recessed space 42 and the second recessed space 52. FIG. 4 depicts a cross-section of the first grasping jaw 15 taken along a plane that extends through the second recessed portion 51 and is substantially perpendicular to the direction of extending disposition of the first grasping jaw 15. Also, in certain examples, the second recessed portion 51 has a width, in other words, a dimension between the side surfaces 55A and 55B of 3 mm or smaller, and has a depth, in other words, a dimension from its opening to the bottom surface 53 of 1 mm or smaller.

On the outer front surface 30 of the first grasping jaw 15, specifically on the bottom surface 53 of the second recessed portion 51, inclined surfaces 57A and 57B are formed. The inclined surfaces 57A and 57B are each inclined relative to the bottom surface 43 of the first recessed portion 41, in other words, the back surface 36. The inclined surface 57A is located on one of opposite sides of the central position P or the central surface in the width direction of the first grasping jaw 15, in other words, the side indicated by the arrow W1, while the inclined surface 57B is located on the other side of the central position P in the width direction of the first grasping jaw 15, in other words, the side indicated by the arrow W2. The bottom surface 53 of the second recessed portion 51 and the bottom surface 43 of the first recessed portion 41 form a substantially T-shape together. The first recessed portion 41 is bent at a distal end thereof into a substantially L-shape from a state, in which the first recessed portion 41 extends along the direction of extending disposition of the first grasping jaw 15, toward the one side in the width direction of the first grasping jaw 15, in other words, the side indicated by the arrow W1, and continues to the inclined surface 57A of the second recessed portion 51. Further, the first recessed portion 41 is also bent at the distal end thereof into a substantially L-shape from the state, in which the first recessed portion 41 extends along the direction of extending disposition of the first grasping jaw 15, toward the other side in the width direction of the first grasping jaw 15, in other words, the side indicated by the arrow W2, and continues to the inclined surface 57B of the second recessed portion 51. The side surfaces 55A and 55B extend at right angle to the bottom surface 43 of the first recessed portion 41 in FIG. 2. However, the side surfaces 55A and 55B may be formed at a desired angle to the bottom surface 43 of the first recessed portion 41 insofar as the liquid can be guided from the bottom surface 43 of the first recessed portion 41 to the bottom surface 53 of the second recessed portion 51.

The inclined surface 57A is arranged extending from the side of the back surface 36 toward the side of the grasping surface 35 as the inclined surface 57A becomes closer to the one side in the width direction of the first grasping jaw 15, in other words, the side indicated by the arrow W1, while the inclined surface 57B is arranged extending from the side of the back surface 36 toward the side of the grasping surface 35 as the inclined surface 57B becomes closer to the other side in the width direction of the first grasping jaw 15, in other words, the side indicated by the arrow W2. In other words, the inclined surfaces 57A and 57B are each arranged extending from the side of the back surface 36 toward the side of the grasping surface 35 as they each become away from the central position Pin the width direction of the first grasping jaw 15, in other words, as they each become closer to the outside. In this embodiment, in a cross-section perpendicular to the direction of extending disposition of the first grasping jaw 15, in other words, directions indicated by the arrow C1 and the arrow C2, the inclined surface 57A is formed in an arcuate shape having a center 01 thereof located on the side of the grasping surface 35 relative to the inclined surface 57A, and the inclined surface 57B is formed in an arcuate shape having a center 02 thereof located on the side of the grasping surface 35 relative to the inclined surface 57B.

The side surface 55A or the wall surface of the second recessed portion 51 is adjacent the distal end sides of the inclined surfaces 57A and 57B or the bottom surface 53. The side surface 55A or the side surface on the distal end side is arranged on the distal end side of the communication part between the first recessed space 42 and the second recessed space 52, in other words, the bottom surface 43 which is used as the flow path surface, and opposes the communication part between the first recessed space 42 and the second recessed space 52. At part on the one side of the central position P in the width direction of the first grasping jaw 15, in other words, the side indicated by the arrow W1, the side surfaces 55A and 55B are each arranged extending along the inclined surface 57A toward the one side away from the central position P in the width direction of the first grasping jaw 15. At part on the other side of the central position P in the width direction of the first grasping jaw 15, in other words, the side indicated by the arrow W2, the side surfaces 55A and 55B are each arranged extending along the inclined surface 57B toward the other side away from the central position P in the width direction of the first grasping jaw 15.

The liquid, which has been supplied toward the distal end side through the first recessed space 42, in other words, along the bottom surface 43 as the flow path surface, flows from the communication part between the first recessed space 42 and the second recessed space 52 into the second recessed space 52. The liquid which has flowed into the second recessed space 52 collides with the side surface 55A or the wall surface of the second recessed portion 51. As a consequence, the flow of the liquid, which is running toward the distal end side along the bottom surface or flow path surface of the first recessed portion 41, changes to flows that run toward the opposite sides away from the central position Pin the width direction of the first grasping jaw 15. In this embodiment, the side surface 55A or the side surface on the distal end side, therefore, functions as a redirecting portion that changes the flow of the liquid, which is running toward the distal end side, to the flows of the liquid that run toward the opposite sides away from the central position Pin the width direction of the first grasping jaw 15.

The redirected liquid is supplied onto the inclined surfaces 57A and 57B. In this embodiment, the liquid is therefore supplied onto the inclined surfaces 57A and 57B by way of the bottom surface 43 of the recessed portion 41 or the flow path surface in or on the back surface 36. The liquid which has been supplied onto the inclined surfaces 57A and 57B flows along the inclined surfaces 57A and 57B toward the opposite sides away from the central position P in the width direction of the first grasping jaw 15. The liquid then flows out or is ejected from each of the inclined surfaces 57A and 57B toward the side of the grasping surface 35, in other words, the side toward which the first grasping jaw 15 is closed. The second recessed portion 51 including the inclined surfaces 57A and 57B is disposed in the distal end portion of the first grasping jaw 15 in this embodiment, so that, at the distal end portion of the first grasping jaw 15, the inclined surfaces 57A and 57B each allow the liquid to flow out toward the side of the grasping surface 35.

On the outer front surface 30 of the first grasping jaw 15, an adjacent surface 61A or a first adjacent surface and another adjacent surface 61B or a second adjacent surface are arranged. The adjacent surface 61A or the first adjacent surface is adjacent to one side of the first recessed portion 41 or the side surface 45A in the width direction of the first grasping jaw 15, and the adjacent surface 61B or the second adjacent surface is adjacent to the other side of the first recessed portion 41 or the side surface 45B in the width direction of the first grasping jaw 15. The adjacent surfaces 61A and 61B are each adjacent to the proximal end side of the second recessed portion 51 or the side surface 55B. On the outer front surface 30 of the first grasping jaw 15, a further adjacent surface 62 or a distal-end-side adjacent surface is arranged adjacent the distal end side of the second recessed portion 51 or the side surface 55A. Owing to the disposition of the recessed portions 41 and 51, the liquid hardly flows out from the bottom surface 43 of the first recessed portion 41 or the flow path surface, and the inclined surfaces 57A and 57B to the adjacent surfaces 61A and 61B, and 62, respectively. Therefore, the liquid which is flowing on the bottom surface 43 and the inclined surfaces 57A and 57B hardly flows out to each of the adjacent surfaces 61A, 61B, and 62, in other words, the outside of the recessed portions 41 and 51.

A description will next be made about functions and advantageous effects of the grasping treatment instrument 2 in this embodiment. The treatment system 1 according to this embodiment is used, for example, in the treatment of the liver, and incision of liver parenchyma, incision of a liver blood vessel, liver hemostasis (coagulation) or the like is performed using the treatment system 1.

Upon performing the incision of liver parenchyma, the end effector 7 is inserted into the abdominal cavity, or body cavity, and the liver parenchyma as the treatment object is grasped between the paired grasping jaws 15 and 16. Here, the grasping surface 35 of the first grasping jaw 15 and a grasping surface of the second grasping jaw 16, in other words, a surface of the second grasping jaw 16, the surface opposing the first grasping jaw 15, each come into contact with the liver parenchyma, or the treatment object, over a range from the proximal end portion to the distal end portion. With the liver parenchyma being grasped between the grasping jaws 15 and 16, the surgeon performs a control input through the control button 18B. When the control input has been performed through the control button 18B, high frequency electric energy is applied across the grasping jaws 15 and 16 from the energy control system 3, and at the same time, electric energy is supplied from the energy control system 3 to the ultrasonic transducer 21. Ultrasonic vibrations then occur at the ultrasonic transducer 21. The ultrasonic vibrations occurred at the ultrasonic transducer 21 are transmitted to the second grasping jaw 16. Consequently, the high frequency electric current flows through the liver parenchyma across the electrode 32 of the first grasping jaw 15 and the second grasping jaw 16, and at the same time, the liver parenchyma is incised with frictional heat generated by the ultrasonic vibrations.

Upon incising a liver blood vessel, on the other hand, the liver blood vessel is grasped between the grasping jaws 15 and 16 in the abdominal cavity. Here, the grasping surface 35 of the first grasping jaw 15 and the grasping surface of the second grasping jaw 16, in other words, the surface of the second grasping jaw 16, the surface opposing the first grasping jaw 15, each come into contact with the blood vessel at a central part thereof in a direction along the longitudinal axis C. With the blood vessel being grasped between the grasping jaws 15 and 16, the surgeon performs a control input through the control button 18B. When the control input has been performed through the control button 18B, a high frequency electric current flows through the blood vessel across the electrode 32 of the first grasping jaw 15 and the second grasping jaw 16 and at the same time, the blood vessel is incised with frictional heat generated by the ultrasonic vibrations, as in the treatment of the incision of the liver parenchyma.

In the treatment upon each of incision of liver parenchyma and incision of a liver blood vessel, heat generated at the heating element 22 may be used instead of ultrasonic vibrations. In this case, specifically, a control input is performed through the control button 18B, whereby high frequency electric energy is supplied to the grasping jaws 15 and 16 from the energy control system 3 and at the same time, electric energy is supplied to the heating element 22 from the energy control system 3 so that heat is generated at the heating element 22.

Upon performing hemostasis in the liver, liver parenchyma is grasped between the grasping jaws 15 and 16 in the abdominal cavity. Here, the grasping surface 35 of the first grasping jaw 15 and the grasping surface of the second grasping jaw 16, in other words, the surface of the second grasping jaw 16, the surface opposing the first grasping surface 15, are each normally brought into contact with the liver parenchyma only at the distal end portion thereof. In other words, the grasping treatment instrument 2 grasps the liver parenchyma at the distal end portions of the grasping jaws 15 and 16. With the grasping jaws 15 and 16 grasping the liver parenchyma, the surgeon performs a control input through the control button 18A, whereby high frequency electric energy is applied across the grasping jaws 15 and 16 from the energy control system 3 and at the same time, a high frequency electric current flows through the liver parenchyma and biological tissue such as blood vessels in the vicinity of the liver parenchyma grasped between the electrode 32 of the first grasping jaw 15 and the second grasping jaw 16. As a consequence, proteins are denatured in the grasped liver parenchyma and the grasped biological tissue such as the blood vessels in the vicinity of the liver parenchyma, so that hemostasis, or coagulation, occurs in and around the grasped liver parenchyma.

In the treatment upon each of incision of liver parenchyma, incision of a liver blood vessel and liver hemostasis, a liquid such as physiological saline is caused to flow out onto the bottom surface 43 of the first recessed portion 41 or the flow path surface through the inside of the feed tube 23, for example, before treatment energy, specifically a high frequency electric current and ultrasonic vibrations are applied to a grasped treatment object or concurrently with the application of the treatment energy. The liquid is then supplied onto the inclined surfaces 57A and 57B of the second recessed portion 51 by way of the bottom surface 43, and subsequently the liquid flows out from the inclined surfaces 57A and 57B toward the side of the grasping surface 35, in other words, the side toward which the first grasping jaw 15 is closed. The liquid which has flowed out from each of the inclined surfaces 57A and 57B then sticks to the grasping surface 35, for example, a surface of the electrode 32, the surface opposing the second grasping jaw 16, by surface tension. Incision, hemostasis, and the like of the treatment object, for example, the liver parenchyma or the liver blood vessel are performed with the liquid stuck on the grasping surface 35, so that sticking of the treatment object to the grasping surface 35, specifically the electrode 32, is prevented and char formation or the like of the treatment object is also prevented.

Also, in this embodiment, as described hereinbefore, the liquid which has flowed onto the back surface 36 or the bottom surface 43 is supplied onto the inclined surface 57A and 57B by way of the bottom surface 43 or the flow path surface, and the liquid then flows out toward the side of the grasping surface 35 from each of the inclined surfaces 57A and 57B. Therefore, the feed route along which the liquid is to be allowed to flow out from each of the inclined surfaces 57A and 57B toward the side of the grasping surface 35 is formed in the outer front surface 30 of the first grasping jaw 15, and no liquid-feeding lumen or the like is disposed inside the first grasping jaw 15. It is, therefore, possible to prevent the occurrence of clogging of biological tissue or the like in the first recessed portion 41 or the first recessed space 42 and the second recessed portion 51 or the second recessed space 52, both of which are used as the feed route. As a consequence, the liquid adequately flows out from each of the inclined surfaces 57A and 57B toward the side of the grasping surface 35, and therefore, the liquid is adequately supplied to the grasped treatment object and its vicinity. Accordingly, the treatment object is treated with the liquid stuck on the grasping surface 35, thereby ensuring treatment performance for the treatment object.

In the treatment for hemostasis in the liver, a treatment object is grasped or held between a distal end portion of the grasping surface 35 of the first grasping jaw 15 and a distal end portion of the second grasping jaw 16 as described hereinbefore. In this embodiment, the inclined surfaces 57A and 57B are arranged on the distal end portion of the first grasping jaw 15, and at the distal end portion of the first grasping jaw 15, the inclined surfaces 57A and 57B each allow the liquid to flow out toward the side of the grasping surface 35. In the treatment for hemostasis in the liver, the liquid which has flowed out from each of the inclined surfaces 57A and 57B is adequately supplied to a vicinity of a treatment object grasped between the distal end portion of the grasping surface 35 of the first grasping jaw 15 and the distal end portion of the second grasping jaw 16. Owing to the supply of an adequate amount of the liquid to the vicinity of the grasped treatment object, sticking of the treatment object to the grasping surface 35 is effectively prevented, and char formation or the like of the treatment object is also effectively prevented.

Also, owing to the disposition of the recessed portions 41 and 51 in this embodiment, the liquid which is flowing on the bottom surface 43 of the first recessed portion 41 and the inclined surfaces 57A and 57B of the second recessed portion 51 hardly flows out to each of the adjacent surfaces 61A, 61B, and 62, in other words, to the outside of the recessed portions 41 and 51. Therefore, a large majority of the liquid which has flowed onto the back surface 36 is allowed to flow out from each of the inclined surfaces 57A and 57B toward the side of the grasping surface 35. In other words, at parts other than the inclined surfaces 57A and 57B, the liquid does not substantially flow out from the side of the back surface 36 toward the side of the grasping surface 35. Therefore, the liquid which has been supplied onto the back surface, specifically the bottom surface 43 as the flow path surface is effectively prevented from flowing to unintended part such as part remote from the treatment object grasped between the distal end portion of the grasping surface 35 of the first grasping jaw 15 and the distal end portion of the second grasping jaw 16. As a consequence, it is possible to effectively prevent a high frequency electric current from flowing, through the liquid, to biological tissue or the like other than the treatment object, for example, liver parenchyma at the part remote from the treatment object. Hence, the high frequency electric current is effectively applied to the treatment object grasped between the distal end portion of the grasping surface 35 of the first grasping jaw 15 and the distal end portion of the second grasping jaw 16, whereby hemostasis or coagulation in the liver can be appropriately achieved using the high frequency electric current.

### Modifications:

Also, in a first modification, a bottom surface 43 or a flow path surface of the first recessed portion 41 and a bottom surface 53 of the second recessed portion 51, the bottom surface 53 including inclined surfaces 57A and 57B, have high hydrophilicity compared with the other parts of the outer front surface 30 such as the adjacent surfaces 61A, 61B, and 62. In certain examples, knurling may be conducted on each of the bottom surfaces 43 and 53 to provide it with higher hydrophilicity. In some other different examples, hydrophilic coating may be applied to each of the bottom surfaces 43 and 53 with a material, which contains silicon dioxide or the like, to provide it with higher hydrophilicity. In certain other different examples, a nanometer-scale corrugation structure or a nanostructure may be formed on each of the bottom surfaces 43 and 53 to provide it with higher hydrophilicity. In further certain examples, knurling, hydrophilic coating and nanometer-scale corrugation may be applied in combination to provide the bottom surfaces 43 and 53 with higher hydrophilicity. Specifically, the high-hydrophilicity bottom surface 43 of the first recessed portion 41 or the flow path surface and the high-hydrophilicity bottom surface 53 of the second recessed portion 51, the bottom surface 53 including the inclined surfaces 57A and 57B, may each be at least one of a surface subjected to knurling, a surface with hydrophilic coating applied thereon, and a surface with a nanometer-scale corrugation structure formed thereon.

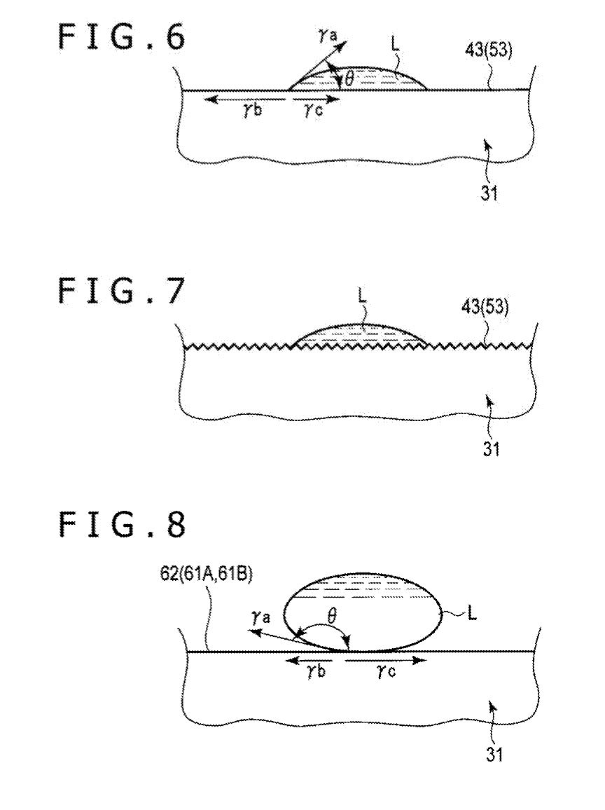

FIG. 6 is a diagram illustrating wetting of the bottom surface 43 of the first recessed portion 41 or the bottom surface 53 of the second recessed portion 51 in the first grasping jaw 15 in this modification. As illustrated in FIG. 6, with a liquid L stuck on the bottom surface 43 or 53 of the first grasping jaw 15, liquid-gas interfacial surface tension γa, solid-gas interfacial surface tension γb and solid-liquid interfacial surface tension γc act as in the case that a liquid, for example, a water droplet or the like has stuck on a solid surface. Now, representing the contact angle of the liquid L to a solid surface, specifically the bottom surface 43 or 53 by θ (0°≤θ≤180°) the following Young's formula (1) can be derived.

[Math. 1]

γb=γa cos θ+γc (1)

On each of the bottom surfaces 43 and 53 having high hydrophilicity, the solid-gas interfacial surface tension γb is high so that the contact angle θ has a value close to 0° such as, for example, 10° or smaller. As the contact angle Θ decreases closer to 0°, the liquid L becomes more sticky to each of the bottom surfaces 43 and 53, in other words, each of the bottom surfaces 43 and 53 becomes more wettable. As a consequence, the bottom surfaces 43 and 53 are provided with high hydrophilicity. Because the liquid L becomes more sticky to each of the bottom surfaces 43 and 53, the liquid L is effectively prevented from flowing out to a vertically lower side from the bottom surfaces 43 and 53 by gravity, for example, even in a posture that the side of the back surface 36 of the first grasping jaw 15, in other words, the side toward which the first grasping jaw 15 is opened becomes the vertically lower side. Therefore, the liquid, which is flowing on the bottom surface 43 of the first recessed portion 41 and the inclined surfaces 57A and 57B of the second recessed portion 51, more hardly flows out to the outside of the recessed portions 41 and 51. As a consequence, the liquid is more adequately supplied onto the inclined surfaces 57A and 57B, leading to an improvement in the supply performance of the liquid to the vicinity of the treatment object grasped between the grasping jaws 15 and 16.

In a second modification illustrated in FIG. 7, a bottom surface 43 of the first recessed portion 41 and a bottom surface 53 of the second recessed portion 51 may each be a surface with a hydrophilic fractal structure formed thereon. In this modification, the bottom surfaces 43 and 53 are each provided with higher hydrophilicity by hydrophilic coating or the like and are each more wettable, in other words, each have a contact angle θ of a value close to 0°, as in the first modification. In this modification, the bottom surfaces 43 and 53 are each formed as a roughened surface by a fractal structure, so that the bottom surfaces 43 and 53 each have a large surface area. If the surface area becomes greater, for example, by forming a roughened surface or the like, hydrophilicity becomes still higher on a high hydrophilicity surface, and water repellency becomes still higher on a high repellency surface. Therefore, by making the surface areas of the bottom surfaces 43 and 53 having high hydrophilicity greater, the bottom surfaces 43 and 53 are provided with still higher hydrophilicity and still higher wettability. Accordingly, the liquid which is flowing on the bottom surface 43 of the first recessed portion 41 and the inclined surfaces 57A and 57B of the second recessed portion 51 becomes still harder to flow out to the outside of the recessed portions 41 and 51. Also, in certain modifications, at least one of knurling, hydrophilic coating, the formation of a nanometer-scale corrugation structure, and the formation of a hydrophilic fractal structure may also be conducted on the side surfaces 45A and 45B of the first recessed portion 41 and the side surfaces 55A and 55B of the second recessed portion 51 in addition to the bottom surfaces 43 and 53. Specifically, in these modifications, in addition to the bottom surfaces 43 and 53, the side surfaces 45A, 45B, 55A, and 55B are also subjected to treatment, processing, or the like to provide them with higher hydrophilicity. As a consequence, in addition to the bottom surfaces 43 and 53, the side surfaces 45A, 45B, 55A, and 55B also have higher hydrophilicity compared with the other parts of the outer front surface 30 such as the adjacent surfaces 61A, 61B, and 62.

Also, in a third modification, adjacent surfaces 61A, 61B, and 62 have higher water repellency compared with the other parts of the outer front surface 30 such as the first recessed portion 41 and the second recessed portion 51. In certain examples, water-repellent coating may be applied to each of the adjacent surfaces 61A, 61B, and 62 with a material, which contains a fluorinated resin, to provide it with higher water repellency.

FIG. 8 is a diagram illustrating wetting of the adjacent surface 61A and 61B or 62 of the first grasping jaw 15 in this modification. As illustrated in FIG. 8, even with a liquid L stuck on each of the adjacent surfaces 61A, 61B, and 62 of the first grasping jaw 15, liquid-gas interfacial surface tension γa, solid-gas interfacial surface tension γb, and solid-liquid interfacial surface tension γc also act as described hereinbefore, and the formula (1) described hereinbefore can be also derived.

On each of the adjacent surfaces 61A, 61B, and 62 having high water repellency, the solid-gas interfacial surface tension γb is small, so that the contact angle θ has a value close to 180° such as, for example, 150° or greater. As the contact angle θ increases closer to 180°, the liquid L becomes less sticky to each of the adjacent surfaces 61A, 61B, and 62, in other words, the liquid L becomes easier to be repelled from each of the adjacent surfaces 61A, 61B, and 62, so that each of the adjacent surfaces 61A, 61B, and 62 becomes less wettable. As a consequence, the adjacent surfaces 61A, 61B, and 62 with high water repellency are formed. Because the liquid L becomes easier to be repelled from each of the adjacent surfaces 61A, 61B, and 62, the liquid becomes still harder to flow out from each of the first recessed portion 41 and the second recessed portion 51 to each of the adjacent surfaces 61A and 61B, and the adjacent surface 62. As a consequence, the liquid is more adequately supplied onto the inclined surfaces 57A and 57B, leading to an improvement in the supply performance of the liquid to the vicinity of the treatment object grasped between the grasping jaws 15 and 16. Further, owing to the higher water repellency of each of the adjacent surfaces 61A, 61B, and 62, fouling matter and the like are hard to stick to each of the adjacent surfaces 61A, 61B, and 62.

Also, in certain modifications, the adjacent surfaces 61A, 61B, and 62 may each be a surface with a water-repellent fractal structure formed thereon. In these modifications, the adjacent surfaces 61A, 61B, and 62 are each provided with higher water repellency by water-repellent coating or the like, and the liquid is easier to be repelled, in other words, the adjacent surfaces 61A, 61B, and 62 each have a contact angle θ of a value close to 180°, as in the third modification. In these modifications, the adjacent surfaces 61A, 61B, and 62 are each formed as a roughened surface by a fractal structure, so that the adjacent surfaces 61A, 61B, and 62 each have a large surface area. When the surface area becomes greater, for example, by forming a roughened surface or the like, hydrophilicity becomes still higher on a high hydrophilicity surface, and water repellency becomes still higher on a high repellency surface, as described hereinbefore. Therefore, by making the surface areas of the adjacent surfaces 61A, 61B, and 62 having high water repellency still greater, the adjacent surfaces 61A, 61B, and 62 are provided with still higher water repellency, and become much less wettable, in other words, become still easier to repel water. Accordingly, the liquid which is flowing on the bottom surface 43 of the first recessed portion 41 and the inclined surfaces 57A and 57B of the second recessed portion 51 become still harder to respectively flow out from the recessed portions 41 and 51 to the adjacent surfaces 61A and 61B, and 62.

Also, in certain modifications, the first modification and the second modification may be combined together. In this case, specifically, the bottom surfaces 43 and 53 are each provided with higher hydrophilicity, for example, by applying hydrophilic coating to or forming a hydrophilic fractal structure on each of the bottom surfaces 43 and 53. Further, the adjacent surfaces 61A, 61B, and 62 are each provided with higher water repellency, for example, by applying water-repellent coating to or forming a water-repellent fractal structure on the adjacent surfaces 61, 61B, and 62. In these modifications, in addition to the bottom surfaces 43 and 53, the side surfaces 45A and 45B of the first recessed portion 41 and the side surfaces 55A and 55B of the second recessed portion 51 may also be provided with higher hydrophilicity by applying hydrophilicity-enhancing treatment, processing, or the like to them such as by applying hydrophilic coating to them.

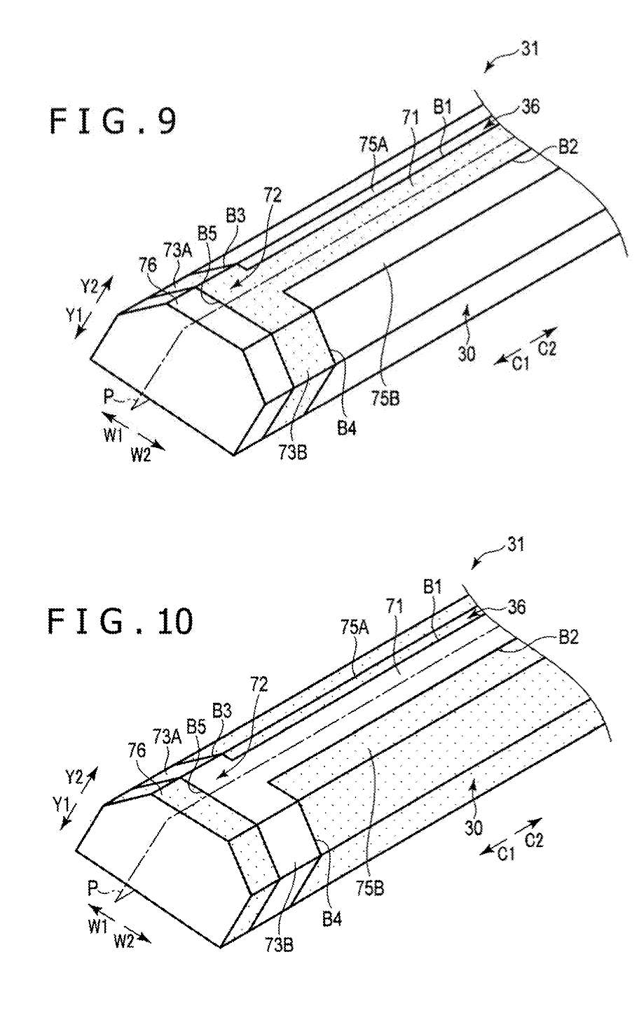

In a fourth modification depicted in FIG. 9, the recessed portion 41 and the recessed portion 51 (see FIG. 5) are not disposed. In the fourth modification, a first flow path surface 71 or a flow path surface is arranged extending along the direction of extending disposition of the first grasping jaw 15, in other words, from the proximal end side toward the distal end side on the back surface 36 of the first grasping jaw 15. The first flow path surface 71 is arranged extending over from the proximal end portion to the distal end portion of the first grasping jaw 15. In this modification, the first flow path surface 71 is located, over substantially the entire length thereof from its proximal end to its distal end, at the central position P and its vicinity in the width direction of the first 2 0 grasping jaw 15. Also, in this modification, a second flow path surface 72 is arranged on the distal end portion of the outer front surface 30 of the first grasping jaw 15, and the first flow path surface 71 is at the distal end thereof in continuation with the second flow path surface 72. Therefore, the second flow path surface 72 is located on the distal end side with respect to the first flow path surface 71. The second flow path surface 72 is arranged extending from the central position P or the central surface toward the opposite sides in the width direction of the first grasping jaw 15, in other words, is arranged extending outwards from the central position P in the width direction of the first grasping jaw 15. The first flow path surface 71 and the second flow path surface 72 form a substantially T-shaped flow path together. The first flow path surface 71 is bent at a distal end thereof into a substantially L-shape from a state, in which the first flow path surface 71 extends along the direction of extending disposition of the first grasping jaw 15, toward the one side in the width direction of the first grasping jaw 15, in other words, the side indicated by the arrow W1, and continues to an inclined surface 73A on the second flow path surface 72, the inclined surface 73A being to be described hereinafter.

The first flow path surface 71 is also bent at the distal end thereof into a substantially L-shape from a state, in which the first flow path surface 71 extends along the direction of extending disposition of the first grasping jaw 15, toward the other side in the width direction of the first grasping jaw 15, in other words, the side indicated by the arrow W2, and continues to an inclined surface 73B on the second flow path surface 72, the inclined surface 73B being to be described hereinafter. The second flow path surface 72 can be formed as desired insofar as the liquid can be guided from the first flow path surface 71 to the second flow path surface 72.

In this modification, the second flow path surface 72 has the inclined surfaces 73A and 73B, which are inclined relative to the first flow path surface 71 or the back surface 36. The inclined surface 73A is located on the one side of the central position P or the central surface in the width direction of the first grasping jaw 15, in other words, the side indicated by the arrow W1, while the inclined surface 73B is located on the other side of the central position P in the width direction of the first grasping jaw 15, in other words, the side indicated by the arrow W2. The inclined surfaces 73A and 73B are each arranged extending from the side of the back surface 36 toward the side of the grasping surface 35 as they each become away from the central position P in the width direction of the first grasping jaw 15, in other words, as they each become closer to the outside.

Also, in this modification, on the outer front surface 30 of the first grasping jaw 15, an adjacent surface or a first adjacent surface 75A, and another adjacent surface or a second adjacent surface 75B are arranged. The adjacent surface or the first adjacent surface 75A is adjacent to one of opposite sides of the first flow path surface 71 in the width direction of the first grasping jaw 15, while the adjacent surface 75B or the second adjacent surface is adjacent to the other side of the first flow path surface 71 in the width direction of the first grasping jaw 15. The adjacent surface 75A has a boundary B1 with the first flow path surface 71, while the adjacent surface 75B has a boundary B2 with the first flow path surface 71. Further, the adjacent surfaces 75A and 75B are adjacent to the proximal end side of the second flow path surface 72 or the proximal end sides of the inclined surfaces 73A and 73B. Therefore, the adjacent surface 75A has a boundary B3 with the second flow path surface 72, specifically the inclined surface 73A, while the adjacent surface 75B has a boundary B4 with the second flow path surface 72, specifically the inclined surface 73B. On the outer front surface 30 of the first grasping jaw 15, an adjacent surface 76, specifically a distal-end-side adjacent surface is also arranged. This adjacent surface, specifically the distal-end-side adjacent surface 76 is adjacent to the distal end side of the second flow path surface 72 or the distal end side of the inclined surfaces 73A and 73B. The adjacent surface 76 has a boundary B5 with the second flow path surface 72.

In this modification, the first flow path surface 71 and the second flow path surface 72 or the inclined surfaces 73A and 73B are each at least one of a surface subjected to knurling, a surface with hydrophilic coating applied thereon, a surface with a nanometer-scale corrugation structure or a nanostructure formed thereon, and a surface with a hydrophilic fractal structure formed thereon, and are each a surface subjected to hydrophilicity-enhancing processing, treatment, or the like as described hereinbefore. Therefore, the flow path surfaces 71 and 72 each have higher hydrophilicity than the other parts of the outer front surface 30. In FIG. 9, the parts of high hydrophilicity on the outer front surface 30 are indicated by a dot pattern.

In this modification, in the first grasping jaw 15, the liquid flows from the port 38 onto the first flow path surface 71 on the back surface 36. Then, the liquid flows from the proximal end side toward the distal end side on the first flow path surface 71. As the first flow path 71 has high hydrophilicity, in other words, is easily wettable, the liquid is effectively prevented from flowing onto the adjacent surface 75A beyond the boundary B1 and also from flowing onto the adjacent surface 75B beyond the boundary B2.

The liquid then flows from the first flow path surface 71 onto the second flow path surface 72. As the second flow path 72 has high hydrophilicity, in other words, is easily wettable, the liquid is effectively prevented from flowing onto the adjacent surface 75A beyond the boundary B3 and also from flowing onto the adjacent surface 75B beyond the boundary B4. Further, the liquid is also effectively prevented from flowing onto the adjacent surface 76 beyond the boundary B5. As a consequence, a flow of the liquid, which is running toward the distal end side along the first flow path surface 71, is changed to flows of the liquid that run toward the opposite sides away from the central position Pin the width direction of the first grasping jaw 15. In this modification, the boundaries B3 to B5, therefore, act as redirecting portions that change the flow of the liquid, which is flowing toward the distal end side, to the flows of the liquid that run toward the opposite sides away from the central position P in the width direction of the first grasping jaw 15.

Then, the liquid is supplied onto the inclined surfaces 73A and 73B, and is allowed to flow along the inclined surfaces 73A and 73B toward the opposite sides away from the central position P in the width direction of the first grasping jaw 15. Subsequently, the liquid flows out, in other words, is ejected from the inclined surfaces 73A and 73B toward the side of the grasping surface 35, in other words, the side toward which the first grasping jaw 15 is closed. In this modification, the second flow path surface 72 which includes the inclined surfaces 73A and 73B is arranged on the distal end portion of the first grasping jaw 15, so that the inclined surfaces 75A and 75B each allow the liquid to flow out toward the side of the grasping surface 35 at the distal end portion of the first grasping jaw 15.

As the liquid is supplied by way of the first flow path surface 71 and the inclined surfaces 73A and 73B on the back surface 36 as described hereinbefore, this modification also exhibits similar functions and advantageous effects as the first embodiment. Therefore, the supply performance of the liquid to the vicinity of the treatment object, for example, liver parenchyma, a liver blood vessel, or the like grasped between the grasping jaws 15 and 16 is also ensured in this modification.

Also, in a fifth modification illustrated in FIG. 10, adjacent surfaces 75A, 75B, and 76 are each at least one of a surface with water-repellent coating applied thereon and a surface with a water-repellent fractal structure formed thereon, and are each a surface subjected to water-repellency-enhancing processing, treatment, or the like as described hereinbefore. Therefore, the adjacent surfaces 75A, 75B, and 76 each have higher water repellency than the other parts of the outer front surface 30. In FIG. 10, the parts of high water repellency on the outer front surface 30 are indicated by a dot pattern.

In this modification, as in the fourth modification, the liquid also flows from the port 38 toward the distal end side on the first flow path 71 on the back surface 36, and the liquid flows onto the second flow path surface 72. Then, the liquid flows out from the inclined surfaces 73A and 73B of the second flow path surface 72 toward the side of the grasping surface 35. As the adjacent surfaces 75A, 75B, and 76 each have high water repellency, in other words, are each easy to repel the liquid in this modification, the liquid is effectively prevented from flowing onto the adjacent surfaces 75A, 75B, and 76 beyond the corresponding boundaries B1 to B5. Therefore, this modification also exhibits similar functions and advantageous effects as the fourth modification. The supply performance of the liquid to the vicinity of the treatment object, for example, liver parenchyma, a liver blood vessel, or the like grasped between the grasping jaws 15 and 16 is also ensured in this modification.

Also, in certain modifications, the fourth modification and the fifth modification may be combined together. In this case, specifically, the flow path surfaces 71 and 72 are each a surface subjected to hydrophilicity-enhancing treatment, processing, or the like, and the adjacent surfaces 75A, 75B, and 76 are each a surface subjected to water-repellency-enhancing treatment, processing, or the like.

In the embodiment, modifications and examples described hereinbefore, the grasping treatment instrument 2 includes the first grasping jaw 15 having the outer front surface 30 exposed to the outside, and the second grasping jaw 16 relative to which the first grasping jaw 15 is configured to be openable and closable. The first grasping jaw 15 has, on the outer front surface 30 thereof, a grasping surface 35 opposing the second grasping jaw 16, a back surface 36 facing an opposite side to the grasping surface 35, and the inclined surfaces 57A and 57B or 73A and 73B which are arranged extending from the side of the back surface 36 toward the side of the grasping surface 35 as the inclined surfaces 57A and 57B or 73A and 73B become away from the central position P in the width direction of the first grasping jaw 15 and inclined relative to the back surface 36. In the first grasping jaw 15, the liquid flows from the liquid inlet portion 38 onto the back surface 36, and the liquid is supplied onto the inclined surfaces 57A and 57B or 73A and 73B by way of the back surface 36. Then, the liquid flows out from the inclined surfaces 57A and 57B or 73A and 73B toward the side of the grasping surface 35.

In sum, the disclosed technology is directed to a grasping treatment instrument comprises a first grasping jaw having opposed respective proximal and distal end portions and an outer front surface. A second grasping jaw is configured to be engaged with first grasping jaw so as to relatively pivot with respect to one another. The first grasping jaw includes a grasping surface formed on the outer front surface and opposing the second grasping jaw. A back surface is formed on the outer front surface and facing an opposite side to the grasping surface. A first recessed portion is formed in the back surface toward a side of the grasping surface and forming a first flow path on which a liquid flows from the proximal end portion toward the distal end portion of the first grasping jaw. A second recessed portion is formed in the outer front surface toward the side of the grasping surface extending in continuation with the first recessed portion and in a direction intersecting the first recessed portion and forming a second flow path on which the liquid flows toward the side of the grasping surface. A liquid port is formed through the proximal end portion of the first grasping jaw so that the liquid flows into the first recessed portion in the back surface. The second recessed portion includes inclined surfaces on the second flow path and the inclined surfaces are formed extending from a side of the back surface toward the side of the grasping surface as the inclined surfaces become away from a central position in a width direction of the first grasping jaw and are inclined relative to the back surface.

The first grasping jaw includes a redirecting portion at the outer front surface that changes a first flow of the liquid that runs from the proximal end portion to the distal end portion along the first flow path to the second flow of the liquid that run along the inclined surfaces toward opposite sides away from the central position in the width direction of the first grasping jaw. The first grasping jaw includes a wall formed facing the proximal end portion and on the distal end side of the first flow path and the wall surface is disposed adjacent to distal end sides of the inclined surfaces and extending along the inclined surfaces toward opposite sides away from the central position in the width direction of the first grasping jaw. The first flow path and the second flow path each of which has at least one of a surface subjected to knurling, a surface with hydrophilic coating applied thereon, a wavy structure surface formed thereon and a surface with a hydrophilic fractal structure formed thereon. The inclined surfaces are formed on the distal end portion of the first grasping jaw. The inclined surfaces each of which is formed in an arcuate shape having a center thereof located on the side of the grasping surface in a cross-section perpendicular to a direction of extending disposition of the first grasping jaw. The first grasping jaw includes an electrode on which the grasping surface is formed and to which high frequency electric energy is supplied.

A grasping treatment instrument comprises a first grasping jaw having opposed respective proximal and distal end portions and an outer front surface. A second grasping jaw is configured to be engaged with first grasping jaw so as to relatively pivot with respect to one another. The first grasping jaw includes a grasping surface formed on the outer front surface and opposing the second grasping jaw. A back surface is formed on the outer front surface and facing an opposite side to the grasping surface. A first flow path is formed on the back surface to direct liquid to flow from the proximal end portion toward the distal end portion. A second flow path is formed on the outer front surface and having inclined surfaces that are formed extending from a side of the back surface toward a side of the grasping surface as the inclined surfaces become away from a central position in a width direction of the first grasping jaw and are inclined relative to the back surface. An adjacent surface is formed adjacent to the first flow path and the second flow path. A liquid port is formed through the proximal end portion of the first grasping jaw so that the liquid flow to the first flow path in the back surface. The first flow path has a surface having higher hydrophilicity than the adjacent surface. The adjacent surface is at least one of a surface with water-repellent coating applied thereon and a surface with a water-repellent fractal structure formed thereon. The adjacent surface includes a first adjacent surface and a second adjacent surface, and a third adjacent surface that is formed adjacent to the second flow path. A first boundary is formed between the first adjacent surface and the first flow path. A second boundary is formed between the second adjacent surface and the first flow path. A third boundary is formed between the first adjacent surface and the second flow path. A fourth boundary is formed between the second adjacent surface and the second flow path. A fifth boundary is formed between the third adjacent surface and the second flow path.

The first grasping jaw includes a redirecting portion at the outer front surface that changes a first flow of the liquid that runs from the proximal end portion toward the distal end portions along the first flow path to a second flow of the liquid that run along the second flow path toward opposite sides away from the central position in the width direction of the first grasping jaw. The first flow path and the second flow path each of which includes at least one of their respective surfaces subjected to knurling, a surface with hydrophilic coating applied thereon, a wavy structure surface formed thereon, and a surface with a hydrophilic fractal structure formed thereon. The adjacent surface is at least one of a surface with water-repellent coating applied thereon and a surface with a water-repellent fractal structure formed thereon. The second flow path is formed on the distal end portion of the first grasping jaw. The second flow path is formed in arcuate shapes each of which has a center thereof located on the side of the grasping surface in a cross-section perpendicular to a direction of extending disposition of the first grasping jaw. The first grasping jaw includes an electrode, on which the grasping surface is formed and to which high frequency electric energy is supplied.

A further aspect of the disclosed technology is directed to a method of using grasping treatment instrument for treating liver parenchyma or a liver blood vessel. The method comprises the steps of grasping the liver parenchyma or the liver blood vessel by respective first and second grasping jaws simultaneously in an abdominal cavity. Next, applying a high frequency electric current to flow across an electrode, which is disposed in the first grasping jaw, and the second grasping jaw to apply the high frequency electric current to the liver parenchyma or the liver blood vessel. Next, supplying a liquid to a back surface, which is formed in an outer front surface of the first grasping jaw that faces an opposite side to the electrode, before or concurrently with applying the high frequency electric current. Finally, directing the liquid to flow by way of a first recessed portion and a second recessed portion that is disposed in continuation with the first recessed portion and extending in a direction intersecting the first recessed portion, and to flow out toward a side of the electrode.

The embodiment, modifications, and examples of the present disclosure have been described above. However, the present disclosure should not be limited to the embodiment, modifications, and examples described hereinbefore, and various modifications are obviously feasible without departing from the spirit of the present disclosure.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. A grasping treatment instrument comprising:
a first grasping jaw having opposed respective proximal and distal end portions, and an outer front surface; and
a second grasping jaw configured to be engaged with first grasping jaw so as to relatively pivot with respect to one another,
wherein the first grasping jaw includes
a grasping surface formed on the outer front surface and opposing the second grasping jaw,
a back surface formed on the outer front surface and facing an opposite side to the grasping surface,
a first recessed portion formed in the back surface toward a side of the grasping surface and forming a first flow path on which a liquid flows from the proximal end portion toward the distal end portion of the first grasping jaw,
a second recessed portion formed in the outer front surface toward the side of the grasping surface extending in continuation with the first recessed portion and in a direction intersecting the first recessed portion and forming a second flow path on which the liquid flows toward the side of the grasping surface, and
a liquid port formed through the proximal end portion of the first grasping jaw so that the liquid flows into the first recessed portion in the back surface.

2. The grasping treatment instrument of claim 1,
wherein the second recessed portion includes inclined surfaces on the second flow path, and the inclined surfaces are formed extending from a side of the back surface toward the side of the grasping surface as the inclined surfaces become away from a central position in a width direction of the first grasping jaw and are inclined relative to the back surface.

3. The grasping treatment instrument of claim 2,
wherein the first grasping jaw includes a redirecting portion at the outer front surface that changes a first flow of the liquid that runs from the proximal end portion to the distal end portion along the first flow path to the second flow of the liquid that run along the inclined surfaces toward opposite sides away from the central position in the width direction of the first grasping jaw.

4. The grasping treatment instrument of claim 2,
wherein the first grasping jaw includes a wall formed facing the proximal end portion and on the distal end side of the first flow path and the wall surface is disposed adjacent to distal end sides of the inclined surfaces and extending along the inclined surfaces toward opposite sides away from the central position in the width direction of the first grasping jaw.

5. The grasping treatment instrument of claim 2,
wherein the first flow path and the second flow path each of which has at least one of a surface subjected to knurling, a surface with hydrophilic coating applied thereon, a wavy structure surface formed thereon and a surface with a hydrophilic fractal structure formed thereon.

6. The grasping treatment instrument of claim 2,
wherein the inclined surfaces are formed on the distal end portion of the first grasping jaw.

7. The grasping treatment instrument of claim 2,
wherein the inclined surfaces each of which formed in an arcuate shape having a center thereof located on the side of the grasping surface in a cross-section perpendicular to a direction of extending disposition of the first grasping jaw.

8. The grasping treatment instrument of claim 1,
wherein the first grasping jaw includes an electrode, on which the grasping surface is formed and to which high frequency electric energy is supplied.

9. A grasping treatment instrument comprising:
a first grasping jaw having opposed respective proximal and distal end portions and an outer front surface; and
a second grasping jaw configured to be engaged with first grasping jaw so as to relatively pivot with respect to one another,
wherein the first grasping jaw includes
a grasping surface formed on the outer front surface and opposing the second grasping jaw,
a back surface formed on the outer front surface and facing an opposite side to the grasping surface,
a first flow path formed on the back surface to direct liquid to flow from the proximal end portion toward the distal end portion,
a second flow path formed on the outer front surface and having inclined surfaces, which are formed extending from a side of the back surface toward a side of the grasping surface as the inclined surfaces become away from a central position in a width direction of the first grasping jaw and are inclined relative to the back surface,
an adjacent surface formed adjacent to the first flow path and the second flow path, and
a liquid port formed through the proximal end portion of the first grasping jaw so that the liquid flow to the first flow path in the back surface, and
the first flow path has a surface having higher hydrophilicity than the adjacent surface.

10. The grasping treatment instrument of claim 9,
wherein the first grasping jaw includes a redirecting portion at the outer front surface that changes a first flow of the liquid, which runs from the proximal end portion toward the distal end portions along the first flow path, to a second flow of the liquid that run along the second flow path toward opposite sides away from the central position in the width direction of the first grasping jaw.

11. The grasping treatment instrument of claim 9,
wherein the first flow path and the second flow path each of which includes at least one of respective surfaces subjected to knurling, a surface with hydrophilic coating applied thereon, a wavy structure surface formed thereon, and a surface with a hydrophilic fractal structure formed thereon.

12. The grasping treatment instrument of claim 9,
wherein the adjacent surface is at least one of a surface with water-repellent coating applied thereon and a surface with a water-repellent fractal structure formed thereon.

13. The grasping treatment instrument of claim 9,
wherein the second flow path is formed on the distal end portion of the first grasping jaw.

14. The grasping treatment instrument of claim 9,
wherein the second flow path is formed in arcuate shapes each of which has a center thereof located on the side of the grasping surface in a cross-section perpendicular to a direction of extending disposition of the first grasping jaw.

15. The grasping treatment instrument of claim 9,
wherein the first grasping jaw includes an electrode, on which the grasping surface is formed and to which high frequency electric energy is supplied.

16. The grasping treatment instrument of claim 9,
wherein the adjacent surface includes a first adjacent surface and a second adjacent surface, and a third adjacent surface is formed adjacent to the second flow path,
a first boundary is formed between the first adjacent surface and the first flow path,
a second boundary is formed between the second adjacent surface and the first flow path,
a third boundary is formed between the first adjacent surface and the second flow path,
a fourth boundary is formed between the second adjacent surface and the second flow path, and
a fifth boundary is formed between the third adjacent surface and the second flow path.

17. A method of using grasping treatment instrument for treating liver parenchyma or a liver blood vessel, the method comprising:
grasping the liver parenchyma or the liver blood vessel by respective first and second grasping jaws simultaneously in an abdominal cavity;
applying a high frequency electric current to flow across an electrode, which is disposed in the first grasping jaw, and the second grasping jaw to apply the high frequency electric current to the liver parenchyma or the liver blood vessel;
supplying a liquid to a back surface, which is formed in an outer front surface of the first grasping jaw that faces an opposite side to the electrode, before or concurrently with applying the high frequency electric current; and
directing the liquid to flow by way of a first recessed portion that is formed in the back surface, and a second recessed portion that is disposed in continuation with the first recessed portion and extending in a direction intersecting the first recessed portion, and to flow out toward a side of the electrode.
